(19) United States
(12) Patent Application Publication
(10) Pub. No.: US 2009/0233976 A1
(43) Pub. Date: Sep. 17, 2009

(54) Antimitotic Rhizoxin Deritivatives of Burkholderia Rhizoxina, Method for Producing Said Derivatives and Use Thereof

(76) Inventors: Kirstin Scherlach, Leipzig (DE); Laila Partida-Martinez, Jena (DE); Christian Hertweck, Leipzig (DE)

Correspondence Address: JORDAN AND HAMBURG LLP, 122 EAST 42ND STREET, SUITE 4000, NEW YORK, NY, 10168, US

(21) Appl. No.: 12/083,165

(22) PCT Filed: Sep. 25, 2006
(86) PCT No.: PCT/DE2006/001708

§ 371 Date: Apr. 25, 2008

(30) Foreign Application Priority Data
Oct. 6, 2005 (DE) 10 2005 048 556.1

(51) Int. Cl. A61K 31/422; C07D 493/04; C12P 17/18; A61P 35/00

(52) U.S. Cl. 514/374; 548/236; 435/119

(57) ABSTRACT

The invention concerns four novel secondary metabolites of the Burkholderia rhizoxina endosymbiont, a method for isolating said compounds from cultures of the bacteria and the use of said substances.

The invention aims at providing novel rhizoxin derivatives with an antimitotic effect. Therefore, the following substances 1-4 are isolated, said substances exhibiting potential antifungal, cytotoxic and antiproliferative properties.

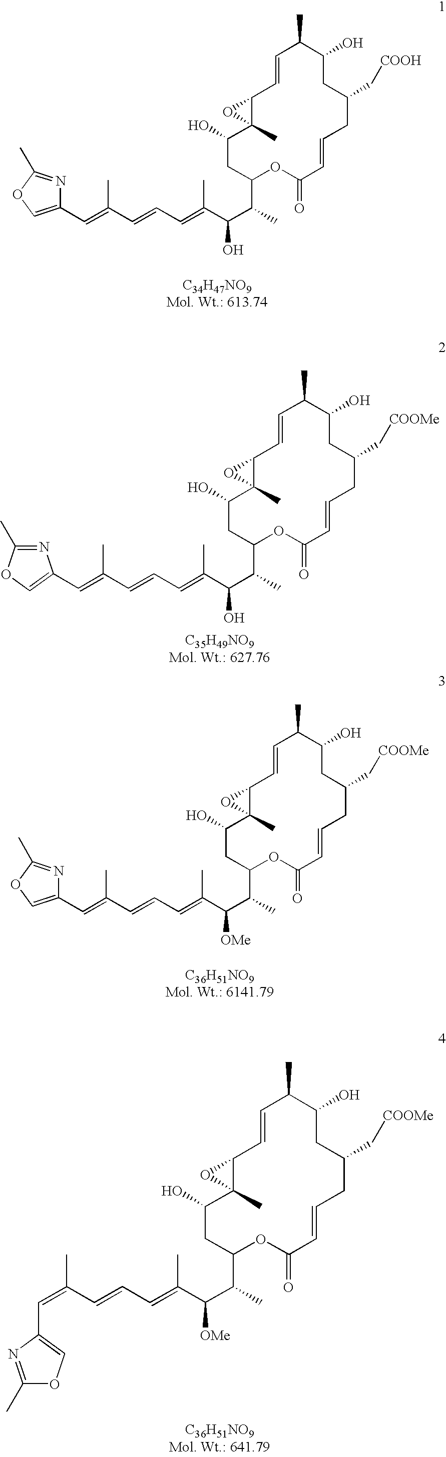

### BACKGROUND OF THE INVENTION

The invention concerns four novel secondary metabolites of the Burkholderia rhizoxina endosymbiont, a method for isolating said compounds from cultures of the bacteria and the use of said substances.

Apart from cardiovascular diseases, malign tumors are the second commonest cause of death in Germany (DKFZ, 2000).

Despite intensive research in the last years, the treatment of some types of cancer is still a great challenge.

Rhizoxin is a macrocyclic polyketide with an antimitotic effect that has been isolated from fungi of the Rhizopus genus (Iwasaki, S. et al. J. Antibiot., 1984, 37, 354-362): It has a high activity against a number of human cancer cell lines, particularly against vincristin-resistent cells, too, and therefore it has become the focus of interest as a potential chemotherapeutic drug. Its effect is based on a bonding to beta-tubulin eukaryotic cells that inhibits the assembling of microtubuli. Recently, we could show that rhizoxin is not formed by Rhizopus but by endosymbionts of the fungus (L. Partida-Martinez and C. Hertweck, Nature, 2005, 437, 884-888). By cultivating the endosymbiont the production of rhizoxin and of derivatives thereof could be considerably increased.

### SUMMARY OF THE INVENTION

It is the object of the present invention to make novel, antimitotic rhizoxin derivatives available and to provide a method for their production. Moreover, the use of said substances is described.

According to the invention, the following antimiotic rhizoxin derivatives are provided:

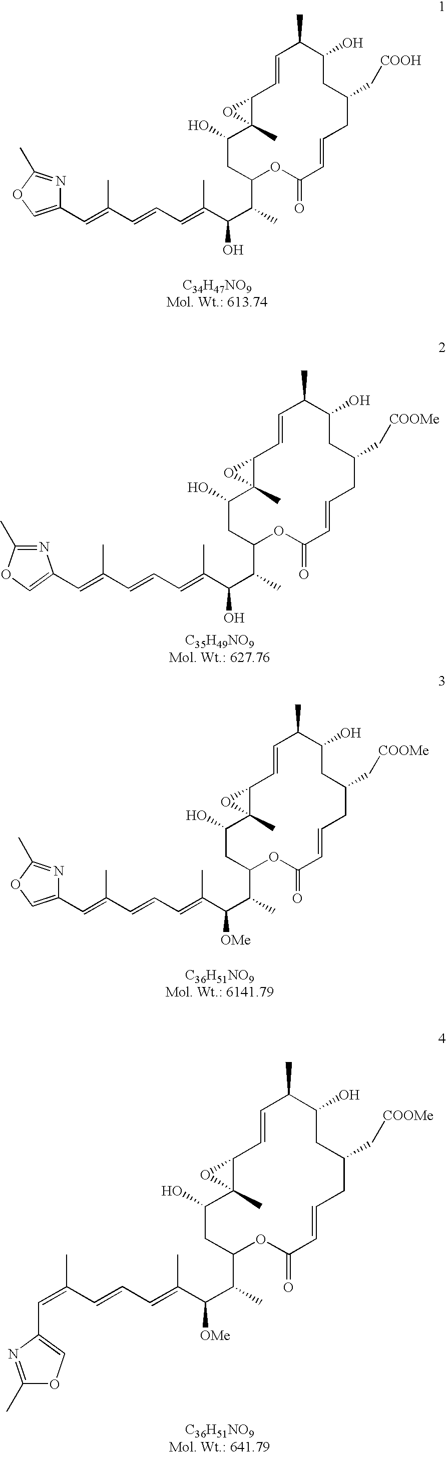

### DETAILED DESCRIPTION OF THE INVENTION

The production of the substances according to the formulas is carried out by the cultivation of the endosymbiotic bacteria strain Burkholderia rhizoxina, the subsequent extraction of the culture and the isolation of the compounds by means of chromatographic methods.

For this purpose, Burkholderia rhizoxina DSM 17360 is cultivated as a shaking culture on a liquid medium and then the grown culture is extracted with organic solvents.

Afterwards, the extract is fractioned via size exclusion chromatography on dextrangels (Sephadex LH-20). The final purification of the substances is performed by means of preparative HPLC by using an RP-18 phase and acetonitril/water-mixtures in the gradient mode.

The structure of the compounds 1-4 is made clear by IR spectroscopy, high-resolution mass spectrometry, and 1D and 2D NMR spectroscopy.

The inventive substances 1-4 show very strong antiproliferative and cytotoxic effects (e.g. for L-929 mouse fibroblast, K-562 human leukemia cells and HeLa human cervix carcinoma line) and an antifungal activity (e.g. against Glomerella cingulata, Penicillium notatum, Fusarium culmorum, Hamigera avellanea, Aspergillus fumigatus). (See Table 1.)

Due to their antiproliferative and cytotoxic properties, the substances 1-4 are very well suited as chemotherapeutic drugs for the treatment of cancer diseases.

Furthermore, the good antifungal effect of the substances 1-4 allows to use them in the therapy of mycoses.

The compounds (1-4) as such can be used in substance or as a pharmaceutical preparation in combination with common additives.

### Exemplary Embodiments

Burkholderia rhizoxina DSM 17360 is cultivated as a shaking culture by means of fermentation on a liquid medium (composition: cornstarch 1%, glycerin 0.5%, yeast extract 1%, corn steep water 1%, CaCO₃ 1%) at 30° C. (4 d). The complete grown culture is extracted with ethyl acetate via stirring and afterwards filtered. This procedure is repeated twice. The combined extracts are dried over sodium sulphate and concentrated. The extract obtained is dissolved in methanol and fractioned via size exclusion chromatography on Sephadex LH-20. The substances 1 through 4 are isolated via preparative HPLC by using an RP-18 phase and acetonitril/water-mixtures (method: MeCN/H₂O 25:75 5 min, then to MeCN/H₂O 80:20 during 35 min, then to MeCN 100% during 5 min, detection at 311 nm).

### Substance 1:

White powder. IR (ATR, solid film) vₘₐₓ/cm⁻¹ 2980, 2926, 2886, 1704, 1654, 1577, 1483, 1380, 1275, 1202, 1153, 1105, 1046, 963, 865, 827, 780, 746, 701. ¹H NMR (300 MHz) and ¹³C NMR (75 MHz) in d-methanol [see Table 2]. (+)-ESI-MS m/z 614 [M+H]⁺, m/z 636 [M+Na]⁺. HRESI-MS: m/z [M+Na]⁺=636.3143 (calculated for C₃₄H₄₇NO₉Na 636.3143)

### Substance 2:

White powder. IR (ATR, solid substance) vₘₐₓ/cm⁻¹ 2977, 2935, 2924, 1705, 1652, 1577, 1437, 1377, 1260, 1202, 1152, 1105, 1048, 1007, 966, 863, 827, 780, 748, 702. ¹H NMR (300 MHz) and ¹³C NMR (75 MHz) in d-chloroform [see Table 2]. (+)-ESI-MS m/z 628 [M+H]⁺, m/z 650 [M+Na]⁺. HRESI-MS: m/z [M+H]⁺=628.3478 (calculated for C₃₅H₅₀NO₉ 628.3486)

### Substance 3:

White powder. IR (ATR, solid substance) vₘₐₓ/cm⁻¹ 2960, 2938, 2928, 1710, 1654, 1577, 1437, 1367, 1275, 1199, 1151, 1108, 1084, 1048, 1008, 971, 862, 827, 753, 706. ¹H NMR (300 MHZ) and ¹³C NMR (75 MHz) in d-chloroform [see Table 2]. (+)-ESI-MS m/z 642 [M+H]⁺, m/z 664 [M+Na]⁺. HRESI-MS: m/z [M+H]⁺=642.3612 (calculated for C₃₆H₅₂NO₉ 642.3637)

### Substance 4:

White powder. IR (ATR, solid substance) vₘₐₓ/cm^{-1 1}H NMR (300 MHz) and ¹³C NMR (75 MHz) in d-methanol [see Table 2]. (+)-ESI-MS m/z 642 [M+H]⁺, m/z 664 [M+Na]⁺. HRESI-MS: m/z [M+Na]⁺=664.3434 (calculated for C₃₆H₅₁NO₉Na 664.3456)

TABLE 1

| | L-929 | K-562 | HeLa |
|---|---|---|---|
| | GI₅₀ | GI₅₀ | CC₅₀ |
| Substance | [μg/ml] | [μg/ml] | [μg/ml] |
| 1 | | 1.5 × 10⁻² | 9 × 10⁻² |
| 2 | 5 × 10⁻² | 3 × 10⁻⁵ | 2.8 × 10⁻⁴ |
| 3 | 5 × 10⁻² | <3 × 10⁻⁵ | <3 × 10⁻⁵ |
| 4 | 1.2 × 10⁻² | <3 × 10⁻⁵ | 2 × 10⁻⁵³ |

The examination of the antiproliferative and cytotoxic properties of the substances 1-4 has been performed via the method described in the literature (H. M. Dahse, B. Schlegel, U. Gräfe, Pharmazie 2001, 56, 489-491). The antifungal activity has been determined via the agar diffusion test. (See Table 2.)

TABLE 2

| | | | | | | | 4 | |
|---|---|---|---|---|---|---|---|---|
| | 1 | 1 | 2 | 2 | 3 | 3 | δH (J | 4 |
| position | δH (J [Hz]) | δC | δH (J [Hz]) | δC | δH (J [Hz]) | δC | [Hz]) | δC |
| 1 | — | 167.0 | — | 165.7 | — | 165.1 | — | 166.9 |
| 2 | 5.78 d (15.6) | 126.2 | 5.70 d (15.6) | 124.6 | 5.67 d (15.6) | 124.9 | 5.77 d (15.6) | 126.2 |
| 3 | 6.79 ddd (15.5, 8.1, | 147.7 | 6.78 ddd (15.5, 9.0, | 147.1 | 6.74 ddd (15.5, 9.0, | 146.4 | 6.78 ddd (15.6, 8.2, | 147.6 |
| | 7.5) | | 6.5) | | 6.6) | | 7.6) | |
| 4 | 2.50 m* | 36.8 | 2.40 m | 37.7 | 2.39 m* | 37.5 | 2.47 m | 36.8 |
| | 2.15 m | | 2.06 m* | | 2.06 m | | 2.13 m | |
| 5 | 2.24 m | 32.8 | 2.21 m | 32.2 | 2.21 m | 32.1 | 2.28 m | 33.0 |
| 5a | 2.50 dd* | 41.3 | 2.52 dd (15.8, 6.9) | 40.5 | 2.51 dd (15.8, 6.8) | 40.4 | 2.52 dd (15.2, 5.6) | 41.2 |
| | 2.35 dd | | 2.35 d (6.9) | | 2.34 dd (15.7, 6.9)* | | 2.36 dd (15.2, 8.2) | |
| 5b | — | 176.6 | — | 173.5 | — | 173.5 | — | 174.8 |
| 6 | 1.75 m | 39.2 | 1.75 dd (14.5, 5.9) | 37.9 | 1.71 m | 37.8 | 1.71 m | 39.1 |
| | 1.10 m | | 1.09 m | | 1.09 m | | 1.05 m | |
| 7 | 3.13 m | 73.9 | 3.20 m | 74.3 | 3.16 m | 74.2 | 3.11 m | 73.9 |
| 8 | 2.02 m | 46.8 | 2.03 m* | 45.5 | 2.02 m | 45.5 | 2.00 m* | 46.8 |
| 8a | 1.02 d (6.0)* | 17.7 | 1.03 d (6.65) | 17.0 | 1.02 d (6.6) | 17.0 | 1.02 d (6.7) | 17.7 |
| 9 | 5.45 dd (15.6; | 142.3 | 5.51 dd (15.6; 9.4) | 141.6 | 5.48 dd (15.6; | 141.4 | 5.43 dd (15.6; 9.3) | 142.3 |
| | 9.2) | | | | 9.4) | | | |
| 10 | 5.16 dd (15.7; | 127.3 | 5.14 dd (15.6; 8.4) | 125.4 | 5.12 dd (15.6; | 125.6 | 15.16 dd (15.6; 8.2) | 127.3 |
| | 8.1) | | | | 8.5) | | | |
| 11 | 3.00 (8.2) | 63.4 | 3.16 d (8.5) | 64.2 | 3.11 d (8.3) | 63.9 | 3.00 d (8.3) | 63.3 |
| 12 | — | 66.3 | — | 65.6 | — | 65.6 | — | 66.2 |
| 12a | 1.29 s | 11.2 | 1.33 s | 11.1 | 1.31 s | 11.1 | 1.29 s | 11.2 |
| 13 | 2.96 dd (11.0; | 79.5 | 3.09 dd (10.2, 3.6) | 78.2 | 3.00 dd (10.8, | 78.3 | 2.94 dd (11.0; 2.6) | 79.5 |
| | 2.7) | | | | 2.8) | | | |
| 14 | 2.03 m* | 34.0 | 1.95 m* | 33.1 | 1.94 m | 31.8 | 1.95 m* | 34.0 |
| | 1.80 m | | 1.89 m | | 1.78 m | | 1.76 m | |
| 15 | 4.76 m | 75.3 | 4.85 m | 74.1 | 4.76 dd (9.9, | 73.3 | 4.75 dd (9.7; 3.5) | 74.9 |
| | | | | | 3.6) | | | |
| 16 | 2.05 m | 41.4 | 1.98 m* | 40.3 | 2.09 m | 39.4 | 2.06 m | 40.6 |
| 16a | 0.99 d (6.5)* | 10.3 | 0.94 d (6.8) | 9.6 | 0.97 d (6.8) | 10.2 | 1.00 d (6.8) | 10.6 |
| 17 | 3.80 d (8.6) | 80.7 | 3.88 d (6.0) | 77.2 | 3.21 d (8.6) | 89.2 | 3.33 d (8.9) | 90.8 |
| 17- | — | — | — | — | 3.13 s | 56.2 | 3.17 s | 56.5 |
| OCH3 | | | | | | | | |
| 18 | — | 140.7 | — | 138.2 | — | 136.3 | — | 138. |
| 18a | 1.89 s | 12.0 | 1.83 s | 12.9 | 1.82 s | 11.6 | 1.84 s | 11.7 |
| 19 | 6.14 d (10.9) | 128.4 | 6.17 d (10.8) | 126.6 | 6.06 d (10.8) | 129.2 | 6.22 d (10.9) | 131.3 |
| 20 | 6.65 dd (15.1; | 125.9 | 6.54 dd (15.1, 10.7) | 124.3 | 6.57 dd (15.1, | 124.1 | 6.71 dd (15.5; | 127.4 |
| | 10.8) | | | | 10.7) | | 11.0) | |
| 21 | 6.40 d (15.2) | 138.3 | 6.35 d (15.2) | 137.6 | 6.34 d (15.2) | 137.6 | 7.27 d (15.3) | 132.8 |
| 22 | — | 139.0 | — | 136.9 | — | 136.9 | — | 137.2 |
| 22a | 2.09 s | 14.7 | 2.11 s | 14.4 | 2.12 s | 14.3 | 2.04 s | 21.0 |
| 23 | 6.21 s | 120.9 | 6.22 s | 120.5 | 6.23 s | 120.7 | 5.78 s | 118.8 |
| 24 | — | 139.5 | — | 138.8 | — | 138.7 | — | 139.1 |
| 25 | 7.79 s | 137.7 | 7.50 s | 135.9 | 7.50 s | 135.9 | 7.74 s | 138. |
| 26 | — | 162.9 | — | 160.9 | — | 160.9 | — | 163.2 |
| 26a | 2.43 s | 13.4 | 2.43 s | 13.8 | 2.43 s | 13.8 | 2.43 s | 13.5 |
| 27 | — | — | 3.68 s | 51.7 | 3.67 s | 51.7 | 3.67 s | 52.0 |

*Partial overlapping of signals

1. A compound of one of the following formulas:

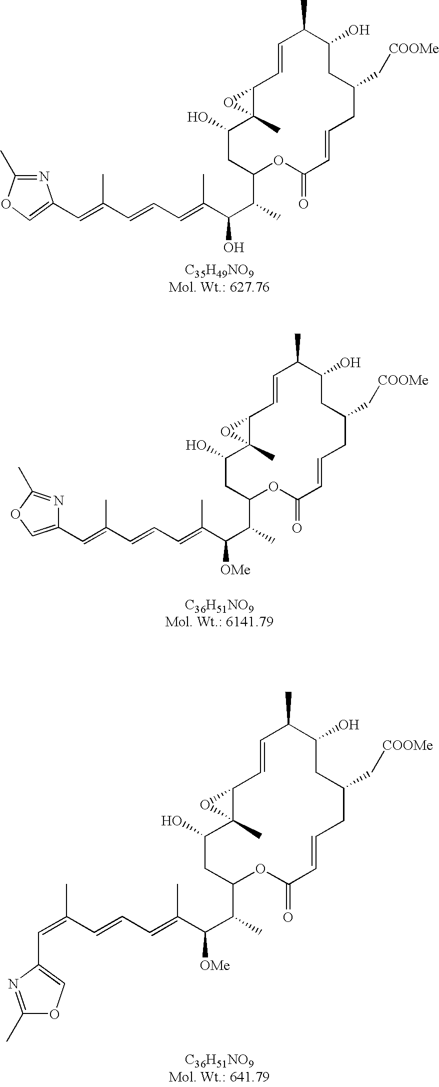

2. A method for producing a compound according to claim 1, comprising cultivating an endosymbiont on a liquid culture medium to produce a culture solution and isolating the compound from the culture solution.

3. The method according to claim 2, wherein the endosymbiont is Burkholderia rhizoxina.

4. The method according to claim 3, wherein the endosymbiont is Burkholderia rhizoxina DSM 17360.

5. A pharmaceutical composition comprising a compound according to claim 1.

6. (canceled)

7. (canceled)

8. (canceled)

9. A method of treating cancer in a person, comprising administering to the person a pharmaceutical composition according to claim 5.

10. A method of treating mycosis in a person, comprising administering to the person a pharmaceutical composition according to claim 5.
